(19) United States
(12) Patent Application Publication
(10) Pub. No.: US 2022/0054578 A1
(43) Pub. Date: Feb. 24, 2022

(54) COMPOSITION AND METHOD FOR ORAL SUPPLEMENT REPELLING BITING INSECTS

(71) Applicant: Life Outside, LLC, Ninety-Six, SC (US)

(72) Inventor: Jeremiah Atkins, Ninety-Six, SC (US)

(21) Appl. No.: 17/410,000

(22) Filed: Aug. 24, 2021

Related U.S. Application Data
(60) Provisional application No. 63/069,251, filed on Aug. 24, 2020.

(51) Int. Cl. A61K 36/8962; A61K 36/899; A61K 36/81; A61K 36/752; A61K 47/02; A61K 47/12; A61K 47/38; A61K 47/10

(52) U.S. Cl. CPC A61K 36/8962; A61K 36/899; A61K 36/81; A61K 47/10; A61K 47/02; A61K 47/12; A61K 47/38; A61K 36/752

(57) ABSTRACT

A composition for repelling or inhibiting biting insects and to prevent game animals, such as deer, wild boar, coyotes and the like, from smelling human odor and methods thereof, wherein the composition comprises from about 20% to 40% garlic extract, from about 5% to about 20% lemongrass extract, from about 1% to about 20% cayenne pepper powder, and from about 10% to about 40% grapefruit concentrated extract with all parts equal to 100%.

### CROSS-REFERENCE TO RELATED APPLICATION

The present patent application/patent claims the benefit of priority of co-pending U.S. Provisional Patent Application No. 63/069,251, filed on Aug. 24, 2020, and entitled “COMPOSITION AND METHOD FOR ORAL SUPPLEMENT REPELLING BITING INSECTS,” the contents of which are incorporated fully herein.

### FIELD OF THE INVENTION

The present invention relates generally to an oral supplement for repelling or inhibiting biting insects, and more generally relates to an oral supplement, composed of natural ingredients, for repelling or inhibiting biting insects and to prevent game animals, such as deer, wild boar, coyotes and the like, from smelling human odor and methods thereof.

### BACKGROUND OF THE INVENTION

Insects, and specifically biting insects, are a part of our natural world and serve an important function in their own right. Any individual that has stepped outdoors has encountered biting insects and the level of discomfort associated with their bite. In addition to the discomfort associated with an insect bite and any associated bump or itchiness, the biting insect could carry or transmit diseases.

There are a number of topical insect repellants on the market. These products are made of chemicals and meant to be applied directly to the skin of a user. On hot summer days, these topical products must be reapplied periodically because sweat and water will wash the products off the skin, rendering them ineffective.

There is a need for an all-natural product that can be ingested to repel biting insects. There is also a need for an all-natural product that is not applied topically to repel biting insects. There is also a need for a product that can repel biting insects, but also serves the dual purpose of masking human odor, preventing animals from smelling the human odor while hunting.

U.S. Pat. No. 7,115,286 titled “COMPOSITIONS AND METHODS FORAN ORALLY ADMINISTERED INHIBITOR OF BITING INSECTS” was filed on Jun. 7, 2004. The composition disclosed in this application discloses The composition of claim 1, comprising: about I 00 to 425 mg of peppermint; about 200 to 850 mg of barley grass; about 200 to 850 mg of lobelia; about 500 to 2000 mg of chlorella; about 20 to 85 mg of watercress; about 20 to 85 mg of alfalfa; about 20 to 85 mg of parsley; about 20 to 85 mg of rice bran; about 75 to 300 mg of thiamin (B-1); about 75 to 300 mg of riboflavin (B-2); about 75 to 300 mg of niacin (B-3); about 75 to 300 mg of panothenic acid (B-5); about 75 to 300 mg of pyridoxine (B-6); about 200 to 800 μg of folic acid (B-9); about 200 to 800 μg of cyanocobalamin (B-12); about 75 to 300 mg of choline; about 75 to 300 mg of inositol; about 75 to 300 μg of cl-biotin; about 575 to 2300 μg of para- aminobenzoic acid; and about 575 to 2300 μg of lecithin. This patent does not disclose the composition of the present invention.

### BRIEF SUMMARY OF THE INVENTION

According to an embodiment of the present invention, the composition of the present invention includes garlic extract, lemongrass extract, cayenne pepper powder, grapefruit concentrated extract.

According to yet another embodiment of the present invention, a capsule containing a composition, the composition includes garlic extract, lemongrass extract, cayenne pepper powder, grapefruit concentrated extract.

According to yet another embodiment of the present invention, a method of repelling biting insects that consists of ingesting a composition that includes garlic extract, lemongrass extract, cayenne pepper powder, grapefruit concentrated extract.

According to yet another embodiment of the present invention, a composition that includes from about 0% to about 100% garlic extract, from about 0% to about 100% lemongrass extract, from about 0% to about 100% cayenne pepper powder, and from about 0% to about 100% grapefruit concentrated extract with all parts equal to 100%.

### DETAILED DESCRIPTION OF THE INVENTION

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

The ranges set forth herein include both numbers at the end of each range and any conceivable number there between, as that is the very definition of a range. It is therefore to be understood that the ranges and limits mentioned herein include all ranges located within the prescribed limits (i.e., subranges). For instance, a range from about 100 to about 200 also includes ranges from 110 to 150, 170 to 190, 153 to 162, and 145.3 to 149.6. Further, a limit of up to about 7 also includes a limit of up to about 5, up to 3, andup to about 4.5, as well as ranges within the limit, such as from about Ito about 5, and from about 3.2 to about 6.5 as examples.

Unless otherwise indicated, all numbers expressing quantities of ingredients used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

As used herein, the terms “comprise”, “comprises”, “containing”; and “has”, “have”, “having”; and “includes”, “include” and “including”; are open-ended transition terms used to transition from a subject recited before the term to one or more elements recited after the term, where the element or elements listed after the transition term are not necessarily the only elements that make up the subject.

The composition of the present invention may be composed of garlic extract, lemongrass extract, cayenne pepper powder, and grapefruit concentrated extract. More specifically, the composition may comprise from about 0% to about 100% garlic extract, from about 0% to about 100% lemongrass extract, from about 0% to about 100% cayenne pepper powder, and from about 0% to about 100% grapefruit concentrated extract with all parts equal to 100%. The composition may be in table form. Alternatively, the composition may be encased in a capsule, such as a gel cap, or the like, for storing the composition in an individual dose and easily allowing a user to ingest the composition. The composition may also be ingested in power form, instead of enclosed in a tablet or capsule. After ingestion, components of the composition are released through the skin pores of the user for repelling or inhibiting biting insects and to prevent game animals, such as deer, wild boar, coyotes and the like, from smelling human odor.

### Garlic Extract

Garlic extract may be harvested from bulb of a garlic plant or the whole garlic plant. The active ingredient in garlic extract for the present invention is Allicin. The crushing or chopping of garlic releases the enzyme alliinase that catalyzes the formation of allicin from S-allyl-L-cysteine sulfoxide (Allin). Allicin rapidly breaks down to forma variety of organosulfur compounds. When used in the present composition and ingested, the allicin of the garlic extract is released through the pores of the user and interferes with the user's natural scent and assists in masking the user from biting insects, such as mosquitos. The preferred garlic extract used in the present inventions is deodorized garlic bulb extract.

### Lemongrass Extract

Lemongrass is a tropical, grassy plant used in cooking and herbal medicine, especially in the Asian culture. Lemongrass extract is extracted from the leaves and stalks of the lemongrass plant and has a citrus scent. The active ingredient in lemongrass extract for the present invention is Citronella. Citronella is a popular natural insect repellant which is generally applied to the skin or used in candle or fragrance form. Lemongrass is safe for human consumption and assists in concealing human odor. The preferred lemongrass extract used in the present invention is West Indian Lemongrass (4:1 extract).

### Cayenne Pepper Powder

Cayenne pepper powder is the dried, pulverized fruit of one or more varieties of cayenne pepper. It is primarily used as a spice to add pungency and flavor to culinary dishes. The active ingredient in cayenne pepper powder for the present invention is capsaicm. Capsaicin is an irritant for many species, including many types of insects. Capsaicin is used as a natural insecticide in many parts of the world, and when consumed, the smell of capsaicin repels biting insects, such as mosquitos. The preferred cayenne pepper used in the present invention is cayenne fruit powder (40,000 HUT/g)

### Grapefruit Concentrated Extract

Grapefruit concentrated extract is made from the seeds and/or pulp of a grapefruit. The active ingredient in grapefruit concentrated extract for the present invention is Nootkatone. Nootkatone is the compound that gives a grapefruit its smell. The Center for Disease Control (CDC) researches have shown that Nootkatone can kill adult mosquitoes, as well as ticks and fleas. According to the CDC, Nootkatone kills mosquitoes, ticks, and fleas by activating the insect's octopamine receptors. These receptors may become hyperactive and vibrate the insect's to death. Nootkatone is an FDA approved food additive. The preferred grapefruit concentrated extract used in the present invention is Grapefruit 4:1 extract (whole fruit).

The garlic extract, lemongrass extract, cayenne pepper powder, and grapefruit are the active ingredients in the composition that are formed into a tablet by a known method. The grapefruit concentrated extract in the present invention inhibits the cytochrome P450 3A4 (CYP3A4) enzyme found in the liver and small intestinal epithelial cells, allowing for higher concentrations of the remaining components within the tablets to be released through the skin pores of the user rather than being broken down in the digestive system. The composition may include the additives such as dicalcium phosphate, vegetable stearic acid, croscarmellose sodium, vegetable magnesium stearate, hydroxypropyl methylcellulose, silicon dioxide, and an aqueous film coating composed of purified water, hydroxypropyl methylcellulose, and vegetable glycerin) and combinations thereof. Generally, the components of the composition are combined with a binder to form a tablet. A tablet may be prepared by the wet granulation method which involves 1) mixing the specific components of the composition using a planetary bowl mixer, ribbon/trough mixer, rotating drum mixer, high-speed mixer or the like until a uniform mixture is achieved consisting of a powder mixture; 2) a binder solution is mixed with the powder mixture to form an adhesive mass that can be granulated; 3) the mass is then screened using a mesh screen to prepare wet granules; and 4) the wet granules are spread evenly on trays and dried in an oven; 5) the dried granules are passed through a screen of smaller size than in step 3; 6) the granules are then separated into coarse and fine granules by shaking in a mesh sieve and a lubricant is passed through the mesh sieve; and 7) the granules are then compressed into tablets. While the wet granulation method has been described herein, any know method to make tablets from a powder composition may be utilized. As mentioned above, the composition may also be enclosed in a gel capsule or consumed in power form.

In manufacturing the composition, the garlic extract, lemongrass extract, cayenne pepper powder, and grapefruit concentrated extract in powder form are combined in a mixing vessel to form a batch. However, the composition can include from about 0% to about 100% garlic extract, from about 0% to about 100% lemongrass extract, from about 0% to about 100% cayenne pepper powder, and from about 0% to about 100% grapefruit concentrated extract with all parts equal to 100%. The composition is then formed into a tablet by known methods, wherein each tablet preferably contains between about 300 to about 600 milligrams of garlic extract, lemongrass extract, and grapefruit concentrated extract and between about 100 to about 300 milligrams of cayenne pepper powder. More preferably between about 350 to about 500 milligrams of garlic extract, lemongrass extract, and grapefruit concentrated extract and between about 100 to about 300 milligrams of cayenne pepper powder.

### EXAMPLE

A 0.375″×0.375″ oblong reddish-orange to reddish-brown tablet was formed. The tablet contained the following components:

TABLE 1

| Active | | | | | |
|---|---|---|---|---|---|
| Ingredients | Label | | Source | Gross | |
| Per Tablet | Claim | | Material | Quantity | |
| Garlic Bulb | 416.00 | mg | Allium | 420.16 | mg |
| Extract | | | sativum | | |
| (deodorized) | | | | | |
| West Indian | 416.00 | mg | West Indian | 168.183 | mg |
| Lemongrass | | | Lemongrass | | |
| (4:1 Extract) | | | (4:1 Extract) | | |
| Cayenne | 208.00 | mg | Capsicum | 93.369 | mg |
| Fruit Powder | | | annuum | | |
| (40,000 HUT/g) | | | | | |
| Grapefruit | 416.00 | mg | Grapefruit | 420.16 | mg |
| 4:1 Extract | | | 4:1 Extract | | |
| (whole fruit) | | | (whole fruit) | | |

TABLE 2

| In-Active Ingredients | Gross Quantity | |
|---|---|---|
| dicalcium phosphate | 162.288 | mg |
| vegetable stearic acid | 90.00 | mg |
| croscarmellose sodium | 50.00 | mg |
| vegetable magnesium | 45.00 | mg |
| stearate | | |
| hydroxypropyl | 30.00 | mg |
| methylcellulose | | |
| silicon dioxide | 20.00 | mg |
| aqueous film coating | 19.00 | mg |
| composed of purified | | |
| water, hydroxypropyl | | |
| methylcellulose, and | | |
| vegetable glycerin) | | |

TABLE 3

| | Percent |
|---|---|
| Ingredient | Composition |
| Garlic Bulb Extract | 27.66% |
| (deodorized) | |
| West Indian Lemongrass | 11.06% |
| (4:1 Extract) | |
| Cayenne Fruit Powder | 6.15% |
| (40,000 HUT/g) | |
| Grapefruit 4:1 Extract | 27.66% |
| (whole fruit) | |
| dicalcium phosphate | 10.68% |
| vegetable stearic acid | 5.92% |
| croscarmellose sodium | 3.29% |
| vegetable magnesium | 2.96% |
| stearate | |
| hydroxypropyl | 1.97% |
| methylcellulose | |
| silicon dioxide | 1.32% |
| aqueous film coating | 1.31% |
| composed of purified | |
| water, hydroxypropyl | |
| methylcellulose, and | |
| vegetable glycerin) | |

The preferable dosages may be 2-3 tablets about 40 minutes to an hour before outdoor activity. For extended outdoor activities, such as camping, the dosage can be 2-3 tablets three times a day or with each meal.

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention and are intended to be covered by the following claims.

What is claimed is:

1. A composition comprising: garlic extract, lemongrass extract, cayenne pepper powder, and grapefruit concentrated extract.

2. The composition according to claim 1, wherein the composition is contained within a tablet.

3. The composition according to claim 1, wherein the composition inhibits insects from biting the user after oral administration of the composition.

4. The composition according to claim 1, wherein the composition masks human odor after oral administration of the composition.

5. The composition according to claim 1, wherein the composition contains between about 300 to about 600 milligrams of garlic extract, lemongrass extract, and grapefruit concentrated extract and between about 100 to about 300 milligrams of cayenne pepper powder.

6. The composition according to claim 1, wherein the composition contains between about 350 to about 500 milligrams of garlic extract, lemongrass extract, and grapefruit concentrated extract and between about 100 to about 300 milligrams of cayenne pepper powder.

7. A composition comprising:
from about 20% to 40% garlic extract, from about 5% to about 20% lemongrass extract, from about 1% to about 20% cayenne pepper powder, and from about 10% to about 40% grapefruit concentrated extract with all parts equal to 100%.

8. The composition according to claim 7, wherein the composition is in tablet form.

9. The composition according to claim 7, wherein the composition inhibits insects from biting the user after oral administration of the composition.

10. The composition according to claim 7, wherein the composition masks human odor after oral administration of the composition.

11. The composition according to claim 7, wherein the composition contains from about 25% to 35% garlic extract, from about 5% to about 15% lemongrass extract, from about 1% to about 10% cayenne pepper powder, and from about 20% to about 35% grapefruit concentrated extract with all parts equal to 100%.

12. The composition according to claim 7, wherein the composition contains from about 25% to 30% garlic extract, from about 8% to about 15% lemongrass extract, from about 3% to about 8% cayenne pepper powder, and from about 25% to about 35% grapefruit concentrated extract with all parts equal to 100%.

13. The composition according to claim 7, wherein the composition includes additives selected from the group dicalcium phosphate, vegetable stearic acid, croscarmellose sodium, vegetable magnesium stearate, hydroxypropyl methylcellulose, silicon dioxide, and an aqueous film coating composed of purified water, hydroxypropyl methylcellulose, and vegetable glycerin) and combinations thereof.

14. A method of inhibiting biting insects, comprising:
providing a composition comprising garlic extract, lemongrass extract, cayenne pepper powder, and grapefruit concentrated extract; and administering the composition orally to a user.

15. The method of inhibiting biting insects according to claim 14, wherein the composition comprises from about 0% to about 100% garlic extract, from about 0% to about 100% lemongrass extract, from about 0% to about 100% cayenne pepper powder, and from about 0% to about 100% grapefruit concentrated extract with all parts equal to 100%.

16. The method of inhibiting biting insects according to claim 14, wherein the composition is in tablet form.

17. The method of inhibiting biting insects according to claim 14, wherein the composition inhibits insects from biting the user after oral administration of the composition.

18. The method of inhibiting biting insects according to claim 14, wherein the composition masks human odor after oral administration of the composition.

19. The method of inhibiting biting insects according to claim 14, wherein the composition contains from about 25% to 35% garlic extract, from about 5% to about 15% lemongrass extract, from about 1% to about 10% cayenne pepper powder, and from about 20% to about 35% grapefruit concentrated extract with all parts equal to 100%.

20. The method of inhibiting biting insects according to claim 14, wherein the composition includes additives selected from the group dicalcium phosphate, vegetable stearic acid, croscarmellose sodium, vegetable magnesium stearate, hydroxypropyl methylcellulose, silicon dioxide, and an aqueous film coating composed of purified water, hydroxypropyl methylcellulose, and vegetable glycerin) and combinations thereof.
